(19) United States
(12) Patent Application Publication
(10) Pub. No.: US 2025/0235152 A1
(43) Pub. Date: Jul. 24, 2025

(54) MULTIMODAL-BASED INTERACTIVE CHATBOT SERVICE METHOD FOR DEGENERATIVE BRAIN FUNCTION DECLINE PREDICTION AND COGNITIVE TRAINING AND APPARATUS FOR THE SAME

(71) Applicant: ELECTRONICS AND TELECOMMUNICATIONS RESEARCH INSTITUTE, Daejeon (KR)

(72) Inventors: Byung Ok KANG, Daejeon (KR); Yoon Hyung KIM, Daejeon (KR); Jeong Uk BANG, Daejeon (KR); Hwa Jeon SONG, Daejeon (KR)

(21) Appl. No.: 18/675,362

(22) Filed: May 28, 2024

(30) Foreign Application Priority Data
Jan. 18, 2024 (KR) 10-2024-0007767

(51) Int. Cl. A61B 5/00; G06F 40/35; G10L 25/66; G16H 50/20; G16H 50/70

(52) U.S. Cl. CPC A61B 5/4803; A61B 5/4064; A61B 5/4088; A61B 5/4842; A61B 5/7267; A61B 5/7275; G06F 40/35; G10L 25/66; G16H 50/20; G16H 50/70

(57) ABSTRACT

A multimodal-based interactive chatbot service method for degenerative brain function decline prediction and cognitive training and an apparatus for the same are provided. A method for supporting prediction for a user's degenerative brain function decline may include outputting first reference content, and at least one system conversation triggering at least one user conversation related to the first reference content to the user through a user interface device; receiving at least one user conversation for the first reference content from the user through the user interface device; and based on at least one of text information corresponding to the at least one user conversation for the first reference content, or voice feature information of the at least one user conversation for the first reference content, acquiring a first prediction result for degenerative brain function decline of the user.

### CROSS-REFERENCE TO RELATED APPLICATION(S)

This application claims the benefit of earlier filing date and right of priority to Korean Application No. 10-2024-0007767, filed on Jan. 18, 2024, the contents of which are all hereby incorporated by reference herein in their entirety.

### BACKGROUND

### 1. Technical Field

The present disclosure relates to degenerative brain function decline prediction and cognitive training, and specifically relates to a multimodal-based interactive chatbot service method for degenerative brain function decline prediction and cognitive training and apparatus for the same.

### 2. Description of Related Art

The brain cognitive disorder cases caused by stroke, forgetfulness, paralysis, etc. including dementia has been increasing not only in the elderly but also in all generations, developing into a social problem. The country is expected to become a super-aged society in 2026 where the elderly population accounts for at least 20% of the total population, and accordingly, the prevalence of aging and degenerative diseases such as dementia and stroke is expected to increase. Nothing is more important than the prevention and preparation of human and financial loss caused by these cognitive disorders at a national level. Degenerative brain function decline such as mild cognitive impairment, dementia, etc. is a disease in which prevention through early prediction comes before anything else, and in particular, it is known that if dementia is detected early and treated continuously, it is effective in delaying the aggravation of symptoms. Accordingly, it is required to develop and improve the medical support technology for predicting, evaluating and diagnosing a cognitive function.

Since cognitive function analysis/evaluation programs performed at the existing dementia care centers and hospitals are conducted by a screening test in a hospital/clinical environment, patients have less access and a difficulty in continuous monitoring. In addition, there are methods for measuring a cognitive function based on a task performance ability such as games, vocabulary tests, etc. with apps available on smart devices, etc., but cognitive function analysis/evaluation is performed according to the predetermined rules for limited tasks, so there are limits in accurately analyzing/evaluating a cognitive function when the user becomes accustomed to performing the task. Accordingly, in order to improve nationwide brain health and raise a medical welfare level by increasing public accessibility, a new method is required to accurately predict and monitor degenerative brain function decline at an early stage by using user-friendly interface devices in daily life, and further to prevent degenerative brain function decline through cognitive function training.

### SUMMARY

The purpose of the present disclosure is to provide a method and an apparatus for degenerative brain function decline prediction and cognitive training based on interactions such as a user conversation, etc. using multimodal technology including images, videos, voices, etc.

The purpose of the present disclosure is to provide a method and an apparatus for degenerative brain function decline prediction and cognitive training based on an image generated by using interactions such as a user conversation, etc.

The technical objects to be achieved by the present disclosure are not limited to the above-described technical objects, and other technical objects which are not described herein will be clearly understood by those skilled in the pertinent art from the following description.

A method for supporting prediction for a user's degenerative brain function decline according to an embodiment of the present disclosure may include outputting first reference content, and at least one system conversation triggering at least one user conversation related to the first reference content to the user through a user interface device; receiving at least one user conversation for the first reference content from the user through the user interface device; and based on at least one of text information corresponding to the at least one user conversation for the first reference content, or voice feature information of the at least one user conversation for the first reference content, acquiring a first prediction result for degenerative brain function decline of the user.

A device for supporting prediction for a user's degenerative brain function decline according to an additional embodiment of the present disclosure may include a memory; a transceiver; and a processor. The processor may be configured to output first reference content and at least one system conversation triggering at least one user conversation related to the first reference content to the user through a user interface device; receive at least one user conversation for the first reference content from the user through the user interface device; and based on at least one of text information corresponding to the at least one user conversation for the first reference content, or voice feature information of the at least one user conversation for the first reference content, acquire a first prediction result for degenerative brain function decline of the user.

In some embodiments of the present disclosure, the method may further include providing the first prediction result to the user through the user interface device.

In some embodiments of the present disclosure, a first user conversation of the at least one user conversation may be associated with a first system conversation of the at least one system conversation, and a second system conversation of the at least one system conversation may be generated or selected based on the first user conversation.

In some embodiments of the present disclosure, the at least one system conversation may be generated or selected based on an understanding-based conversation task for the first reference content.

In some embodiments of the present disclosure, the first prediction result may be acquired based on at least one of application of a patient group model and a normal group model learned based on multimodal data including content and a text to the first reference content and text information corresponding to the at least one user conversation for the first reference content; application of a patient group model and a normal group model learned based on linguistic feature data to text information corresponding to the at least one user conversation for the first reference content; or application of a patient group model and a normal group model learned based on voice feature data to voice feature information of the at least one user conversation for the first reference content.

In some embodiments of the present disclosure, the method may further include outputting second reference content selected based on a first prediction result for degenerative brain function decline of the user, and at least one system conversation triggering at least one user conversation related to the second reference content to the user through the user interface device; receiving at least one user conversation for the second reference content from the user through the user interface device; and based on at least one of text information corresponding to the at least one user conversation for the second reference content, or voice feature information of the at least one user conversation for the second reference content, acquiring a second prediction result for degenerative brain function decline of the user.

In some embodiments of the present disclosure, the method may further include providing the second prediction result to the user through the user interface device.

In some embodiments of the present disclosure, the content may include at least one of an image, a video, a text, a voice, or a sound.

A method for supporting prediction for a user's degenerative brain function decline according to an additional embodiment of the present disclosure may include outputting first reference content, and at least one system conversation triggering at least one user conversation related to the first reference content to the user through a user interface device; receiving at least one user conversation for the first reference content from the user through the user interface device; and based on first generated content generated based on the at least one user conversation for the first reference content, acquiring a first prediction result for degenerative brain function decline of the user.

A device for supporting prediction for a user's degenerative brain function decline according to an additional embodiment of the present disclosure may include a memory; a transceiver; and a processor. The processor may be configured to output first reference content, and at least one system conversation triggering at least one user conversation related to the first reference content to the user through a user interface device; receive at least one user conversation for the first reference content from the user through the user interface device; and based on first generated content generated based on the at least one user conversation for the first reference content, acquire a first prediction result for degenerative brain function decline of the user.

In some embodiments of the present disclosure, the method may further include providing the first prediction result to the user through the user interface device.

In some embodiments of the present disclosure, a first user conversation of the at least one user conversation may be associated with a first system conversation of the at least one system conversation, and a second system conversation of the at least one system conversation may be generated or selected based on the first user conversation.

In some embodiments of the present disclosure, the at least one system conversation may be generated or selected based on an understanding-based conversation task for the first reference content.

In some embodiments of the present disclosure, the method may further include outputting first generated content generated based on the at least one user conversation for the first reference content to the user through the user interface device.

In some embodiments of the present disclosure, the first prediction result may be acquired through application of a patient group model and a normal group model learned based on content data for the first reference content, and first generated content generated based on the at least one user conversation for the first reference content.

In some embodiments of the present disclosure, the method may further include outputting second reference content selected based on a first prediction result for degenerative brain function decline of the user, and at least one system conversation triggering at least one user conversation related to the second reference content to the user through the user interface device; receiving at least one user conversation for the second reference content from the user through the user interface device; and based on second generated content generated based on the at least one user conversation for the second reference content, acquiring a second prediction result for degenerative brain function decline of the user.

In some embodiments of the present disclosure, the method may further include providing the second prediction result to the user through the user interface device.

In some embodiments of the present disclosure, the method may further include outputting second generated content generated based on the at least one user conversation for the second reference content to the user through the user interface device.

In some embodiments of the present disclosure, the content may include at least one of an image, a video, a text, a voice, or a sound.

The features briefly summarized above with respect to the present disclosure are just an exemplary aspect of a detailed description of the present disclosure described below, and do not limit a scope of the present disclosure.

According to the present disclosure, a method and an apparatus for degenerative brain function decline prediction and cognitive training based on interactions such as a user conversation, etc. using multimodal technology including images, videos, voices, etc. may be provided.

According to the present disclosure, a method and an apparatus for degenerative brain function decline prediction and cognitive training based on an image generated by using interactions such as a user conversation, etc. may be provided.

Effects achievable by the present disclosure are not limited to the above-described effects, and other effects which are not described herein may be clearly understood by those skilled in the pertinent art from the following description.

### BRIEF DESCRIPTION OF DRAWINGS

FIG. 1 shows an example of a system for supporting degenerative brain function decline prediction and cognitive training according to the present disclosure.

FIG. 2 shows an example of a method for supporting degenerative brain function decline prediction and cognitive training according to the present disclosure.

FIG. 3 shows an example of a conversation voice/text-based degenerative brain function decline prediction operation using content according to the present disclosure.

FIG. 4 shows an example of conversation voice/text-based degenerative brain function decline prediction according to the present disclosure.

FIG. 5 shows an example of a generated content-based degenerative brain function decline prediction operation according to the present disclosure.

FIG. 6 shows an example of generated content-based degenerative brain function decline prediction according to the present disclosure.

FIG. 7 shows an example of an interactive cognitive function training/improvement method using content according to the present disclosure.

### DETAILED DESCRIPTION

As the present disclosure may make various changes and have multiple embodiments, specific embodiments are illustrated in a drawing and are described in detail in a detailed description. But, it is not to limit the present disclosure to a specific embodiment, and should be understood as including all changes, equivalents and substitutes included in an idea and a technical scope of the present disclosure. A similar reference numeral in a drawing refers to a like or similar function across multiple aspects. A shape and a size, etc. of elements in a drawing may be exaggerated for a clearer description. A detailed description on exemplary embodiments described below refers to an accompanying drawing which shows a specific embodiment as an example. These embodiments are described in detail so that those skilled in the pertinent art can implement an embodiment. It should be understood that a variety of embodiments are different each other, but they do not need to be mutually exclusive. For example, a specific shape, structure and characteristic described herein may be implemented in other embodiment without departing from a scope and a spirit of the present disclosure in connection with an embodiment. In addition, it should be understood that a position or an arrangement of an individual element in each disclosed embodiment may be changed without departing from a scope and a spirit of an embodiment. Accordingly, a detailed description described below is not taken as a limited meaning and a scope of exemplary embodiments, if properly described, are limited only by an accompanying claim along with any scope equivalent to that claimed by those claims.

In the present disclosure, a term such as first, second, etc. may be used to describe a variety of elements, but the elements should not be limited by the terms. The terms are used only to distinguish one element from other element. For example, without getting out of a scope of a right of the present disclosure, a first element may be referred to as a second element and likewise, a second element may be also referred to as a first element. A term of and/or includes a combination of a plurality of relevant described items or any item of a plurality of relevant described items.

When an element in the present disclosure is referred to as being “connected” or “linked” to another element, it should be understood that it may be directly connected or linked to that another element, but there may be another element between them. Meanwhile, when an element is referred to as being “directly connected” or “directly linked” to another element, it should be understood that there is no another element between them.

As construction units shown in an embodiment of the present disclosure are independently shown to represent different characteristic functions, it does not mean that each construction unit is composed in a construction unit of separate hardware or one software. In other words, as each construction unit is included by being enumerated as each construction unit for convenience of a description, at least two construction units of each construction unit may be combined to form one construction unit or one construction unit may be divided into a plurality of construction units to perform a function, and an integrated embodiment and a separate embodiment of each construction unit are also included in a scope of a right of the present disclosure unless they are beyond the essence of the present disclosure.

A term used in the present disclosure is just used to describe a specific embodiment, and is not intended to limit the present disclosure. A singular expression, unless the context clearly indicates otherwise, includes a plural expression. In the present disclosure, it should be understood that a term such as “include” or “have”, etc. is just intended to designate the presence of a feature, a number, a step, an operation, an element, a part or a combination thereof described in the present specification, and it does not exclude in advance a possibility of presence or addition of one or more other features, numbers, steps, operations, elements, parts or their combinations. In other words, a description of “including” a specific configuration in the present disclosure does not exclude a configuration other than a corresponding configuration, and it means that an additional configuration may be included in a scope of a technical idea of the present disclosure or an embodiment of the present disclosure.

Some elements of the present disclosure are not a necessary element which performs an essential function in the present disclosure and may be an optional element for just improving performance. The present disclosure may be implemented by including only a construction unit which is necessary to implement essence of the present disclosure except for an element used just for performance improvement, and a structure including only a necessary element except for an optional element used just for performance improvement is also included in a scope of a right of the present disclosure.

Hereinafter, an embodiment of the present disclosure is described in detail by referring to a drawing. In describing an embodiment of the present specification, when it is determined that a detailed description on a relevant disclosed configuration or function may obscure a gist of the present specification, such a detailed description is omitted, and the same reference numeral is used for the same element in a drawing and an overlapping description on the same element is omitted.

In the present disclosure, various examples of a degenerative brain function decline prediction and cognitive training method and device based on an interaction (e.g., a conversation) using multimodal technology are described.

In examples of the present disclosure, a method is described for predicting degenerative brain function decline (e.g., mild cognitive impairment, dementia, etc.) based on an everyday conversation using various types of content presented/output to a user by using a user interface device to which a user may be easily accessible in daily life (e.g., a smartphone, a tablet PC, a smart TV, an interactive robot, etc.). In addition, examples of the present disclosure include an interactive cognitive function training/improvement method based on a degenerative brain function decline prediction method.

Here, a user may be mainly the elderly, but is not limited thereto, and may be a general user who wishes to check degenerative brain function decline. In addition, content may mainly include an image or a video, and may further include various multimedia contents such as a text, a voice, a sound, etc. A situation presented to a user through content may include, for example, a situation related to daily life that a user may easily understand, but is not limited thereto. In other words, content for a variety of situations without a substantive limit may be presented to a user.

In addition, an everyday conversation using content may include, for example, a conversation for confirming a user's understanding of content presented/output to a user (e.g., a question and an answer about a situation included in corresponding content, a situation description). A conversation may include a system conversation output by a system through a user interface device, and a user conversation input by a user through a user interface device. A system conversation and a user conversation may be interrelated or exchanged sequentially. A combination of this system conversation and user conversation may be referred to as an understanding-based conversation task for content. In other words, a system conversation may be automatically generated based on an understanding-based conversation task for content to derive a user conversation.

Additional examples of the present disclosure may include a method for generating new content based on a user's conversation/vocalization that describes a situation about content presented/output to a user and predicting degenerative brain function decline by comparing generated content with presented content, and an interactive cognitive function training/improvement method related thereto.

In this way, through various examples of the present disclosure, a degenerative brain function decline prediction and cognitive training method targeting content provided with almost no restrictions may be provided.

In examples of the present disclosure, exchange of a system conversation and a user conversation may be referred to as a chatbot service, but a scope of the present disclosure is not limited by such name, and may include various methods for exchanging a system conversation and a user conversation.

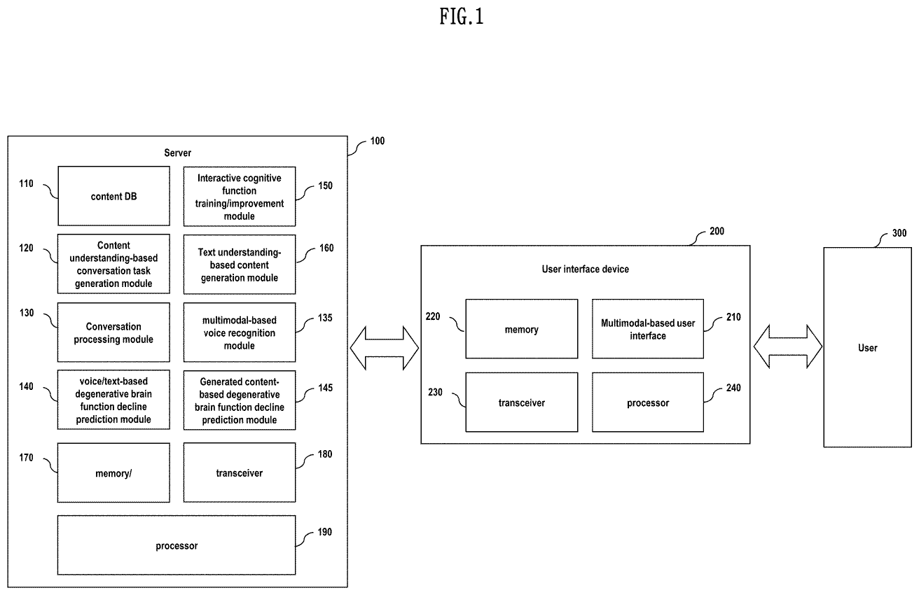

FIG. 1 shows an example of a system for supporting degenerative brain function decline prediction and cognitive training according to the present disclosure.

A system may include a server 100, a user interface device 200, and a user 300.

A server 100 may include content database (DB) 110, a content understanding-based conversation task generation module 120, a conversation processing module 130, a multimodal-based voice recognition module 135, a voice/text-based degenerative brain function decline prediction module 140, a generated content-based degenerative brain function decline prediction module 145, an interactive cognitive function training/improvement module 150, and a text understanding-based content generation module 160.

Furthermore, a server 100 may further include a memory 170, a transceiver 180, and a processor 190. A memory 170 may be included in a processor 190 or may be configured separately. A memory 170 may store an instruction that makes a server 100 perform an operation when executed by a processor 190. A transceiver 180 may transmit and/or receive a signal, data, etc. exchanged by a server 100 with other devices. Among elements of a server 100, at least one of elements other than a memory 170, a transceiver 180, and a processor 190 may not be included according to various examples of the present disclosure, and other elements not shown in FIG. 1 may be included in a server 100.

A processor 190 may configure a server 100 to perform an operation for content storage and maintenance, content generation, conversation task generation, conversation processing, voice recognition, degenerative brain function decline prediction, and cognitive function training/improvement according to various examples of the present disclosure. For example, a server 100 may be configured as a set of modules that perform each function shown in FIG. 1. A module may be configured in a form of hardware and/or software.

A user interface device 200 may include a multimodal-based user interface 210 that outputs content, a system conversation, a prediction result for degenerative brain function decline, etc. to a user 300 and receives a user conversation, a user control signal, etc. from a user 300. For example, a multimodal-based user interface 210 may include a display that outputs an image/a video/a text, a speaker that outputs a voice/a sound, a microphone that acquires a user's voice information, a camera that acquires a user's motion information, etc.

In addition, a user interface device 200 may further include a memory 220, a transceiver 230, and a processor 240. A memory 220 may be included in a processor 240 or may be configured separately. A memory 220 may store an instruction that makes a user interface device 200 perform an operation when executed by a processor 240. A transceiver 230 may transmit and/or receive a signal, data, etc. exchanged by a user interface device 200 with other devices. Other elements not shown in FIG. 1 may be included in a user interface device 200. For example, a user interface device 200 may include a smartphone, a smart TV, a tablet PC, an interactive robot, etc.

A processor 240 may configure a user interface device 200 to perform an operation that supports prediction for a user's degenerative brain function decline according to various examples of the present disclosure. Although not shown in FIG. 1, a processor 240 may be configured as a set of modules that perform each function. A module may be configured in a form of hardware and/or software.

For example, a processor 240 may be configured to output reference content, and at least one system conversation triggering at least one user conversation related to reference content to a user 300 through a multimodal-based user interface 210; receive at least one user conversation for reference content from a user 300 through a multimodal-based user interface 210; and based on at least one of text information corresponding to at least one user conversation for reference content, voice feature information of at least one user conversation for reference content, or generated content generated based on at least one user conversation for reference content, acquire a prediction result for degenerative brain function decline of a user 300. For example, reference content and at least one system conversation may be transmitted from a server 100 to a user interface device 200. At least one user conversation may be transmitted from a user interface device 200 to a server 100. Prediction for degenerative brain function decline based on text information/voice feature information/generated content may be performed in a server 100, and a corresponding prediction result may be acquired by a user interface device 200 provided from a server 100. In addition, a prediction result may be provided to a user 300 from a user interface device 200.

Additionally or alternatively, a user interface device 200 may include at least one function module that corresponds to (e.g., assists a function of a corresponding function module in a server 100) or replaces at least one of a variety of function modules of a server 100 shown in FIG. 1. For example, a multimodal-based voice recognition module 135 is included in a user interface device 200, not a server 100, so text information and/or voice feature data corresponding to a user conversation may be acquired by a user interface device 200 and transmitted to a server 100. A server 100 may generate/select a system conversation based on a conversation task, generate generated content, extract a linguistic feature and extract a voice feature based on text information and/or voice feature information received from a user interface device 200. Other than a multimodal-based voice recognition module 135, function module(s) corresponding to or replacing function module(s) of a server 100 shown in FIG. 1 may be included in a user interface device 200. In examples described later, an operation performed by a function module of a server 100 is mainly described as the representative example of the present disclosure, but an operation performed by a corresponding function module included in a user interface device 200 is also included in the scope of the present disclosure. In other words, in the present disclosure, a function of a server 100 may be inherent in a server 100 and/or a user interface device 200, and examples described below are mainly described through input and output of a multimodal-based interface 210 like information/data/signal exchanged between a user interface device 200 and a user 300.

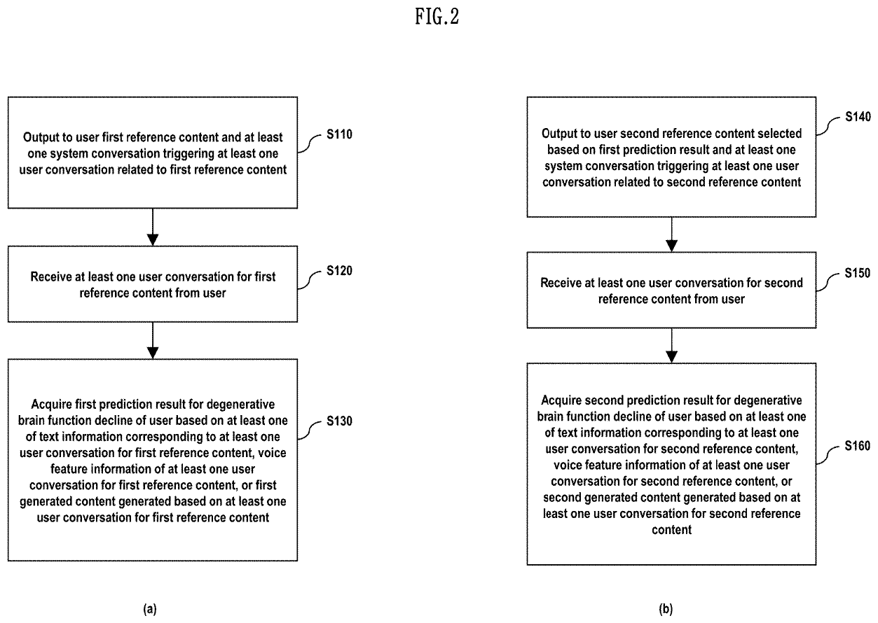

FIG. 2 shows an example of a method for supporting degenerative brain function decline prediction and cognitive training according to the present disclosure.

FIG. 2(a) may correspond to an example of a method for supporting prediction for a user's degenerative brain function decline.

In S110, a user interface device 200 may output first reference content to a user 300 and may also output at least one system conversation triggering at least one user conversation related to first reference content to a user 300.

Reference content may correspond to content that is generated/selected by a server 100 and presented/output to a user 300 through a user interface device 200. At least one system conversation may be generated or selected by a server 100 based on an understanding-based task for reference content and provided to a user interface device 200. For example, a first user conversation among at least one user conversation may be associated with a first system conversation among at least one system conversation. In addition, a second system conversation among at least one system conversation may be generated/selected based on a first user conversation. In this way, a system conversation and a user conversation may be interrelated or exchanged sequentially.

In S120, a user interface device 200 may receive at least one user conversation for first reference content from a user 300.

At least one user conversation may be transmitted to a server 100, so a text corresponding to a user conversation may be acquired through voice recognition in a server 100, and a subsequent system conversation based on a user conversation may be generated/selected by a server 100. In addition, first generated content may be generated/selected by a server 100 based on at least one user conversation for first reference content.

In S130, a user interface device 200 may acquire a first prediction result regarding a user's degenerative brain function decline.

A first prediction result may be generated by a server 100 based on i) text information corresponding to at least one user conversation for first reference content, ii) voice feature information of at least one user conversation for first reference content, and/or iii) first generated content generated based on at least one user conversation for first reference content.

For example, a first prediction result may be generated based on application of a patient group model and a normal group model learned based on multimodal data including content and a text to first reference content and text information corresponding to at least one user conversation for first reference content.

Additionally or alternatively, a first prediction result may be generated based on application of a patient group model and a normal group model learned based on linguistic feature data to text information corresponding to at least one user conversation for first reference content.

Additionally or alternatively, a first prediction result may be generated based on application of a patient group model and a normal group model learned based on voice feature data to voice feature information of at least one user conversation for first reference content.

Additionally or alternatively, a first prediction result may be generated based on application of a patient group model and a normal group model learned based on content data to first generated content generated based on at least one user conversation for first reference content.

In this way, a first prediction result generated by a server 100 may be acquired by a user interface device 200 and provided/output to a user 300. In addition, first generated content generated by a server 100 may be provided/output to a user 300 through a user interface device 200.

FIG. 2(b) may correspond to an example of a method for supporting cognitive function training based on a prediction result.

In S140, a user interface device 200 may output second reference content selected based on a first prediction result to a user 300, and also may output at least one system conversation triggering at least one user conversation related to second reference content to a user 300.

For example, a first prediction result may correspond to a cognitive function level of a user 300 determined according to an exemplary method described in FIG. 2(a). In other words, second reference content selected based on a first prediction result may correspond to reference content that matches a cognitive function level of a user 300. In this way, second reference content may correspond to content that is selected by a server 100 and is presented/output to a user 300 through a user interface device 200 for cognitive function training/improvement of a user 300.

A specific feature for at least one system conversation and at least one user conversation for second reference content corresponds to a specific feature for at least one system conversation and at least one user conversation for first reference content described in S110 of FIG. 2(a), so an overlapping description is omitted.

In S150, a user interface device 200 may receive at least one user conversation for second reference content from a user 300.

A specific feature in which at least one user conversation for second reference content is transmitted to a server 100 and processed corresponds to a specific feature for at least one user conversation for first reference content described in S120 of FIG. 2(a), so an overlapping description is omitted.

In S160, a user interface device 200 may acquire a second prediction result regarding a user's degenerative brain function decline.

A second prediction result may be generated by a server 100 based on i) text information corresponding to at least one user conversation for second reference content, ii) voice feature information of at least one user conversation for second reference content, and/or iii) second generated content generated based on at least one user conversation for second reference content.

A specific feature in which a second prediction result is generated by a server 100 and acquired by a user interface device 200 corresponds to a specific feature for a first prediction result described in S130 of FIG. 2(a), so an overlapping description is omitted.

In this way, a second prediction result generated by a server 100 may be acquired by a user interface device 200 and provided/output to a user 300. In addition, second generated content generated by a server 100 may be provided/output to a user 300 through a user interface device 200.

In addition to an example of FIG. 2, generation/output of third reference content and generation/acquisition of a third prediction result through a system conversation and a user conversation therefor may be performed. In other words, generation/output of reference content and generation/acquisition of a prediction result for a user's degenerative brain function decline through a system conversation and a user conversation therefor may be performed multiple times for cognitive function training/improvement of a user 300.

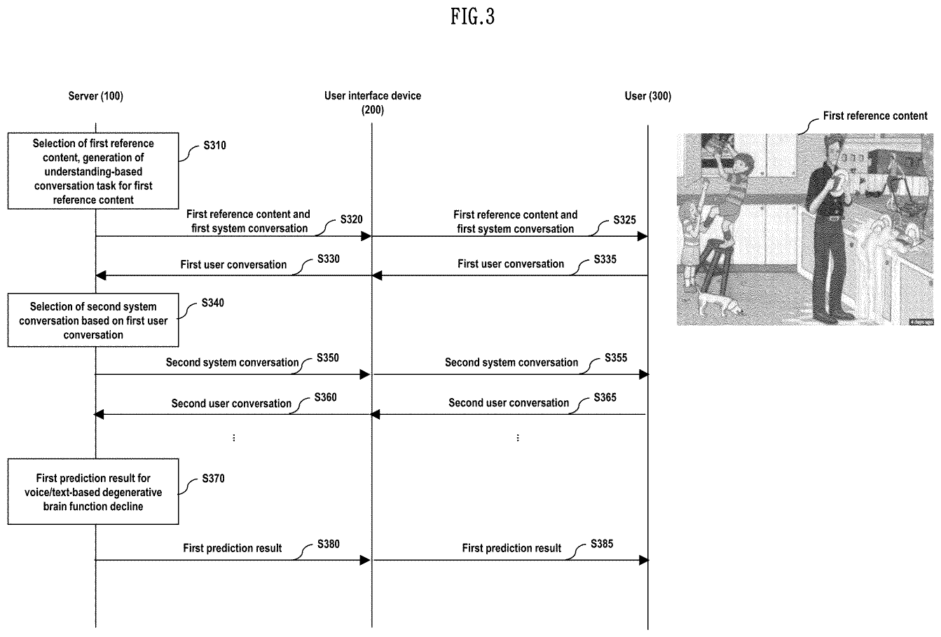

FIG. 3 shows an example of a conversation voice/text-based degenerative brain function decline prediction operation using content according to the present disclosure.

A server 100 may select first reference content and generate an understanding-based conversation task for first reference content S310. An image corresponding to an example of selected first reference content is shown at a top-right position of FIG. 3.

In this process, content DB 110 and a content understanding-based conversation task generation module 120 of a server 100 may be involved. For example, a content understanding-based conversation task generation module 120 may target first reference content selected from content DB 110 and generate a content understanding-based conversation task for degenerative brain function decline prediction. A content understanding-based conversation task may correspond to a conversation task that performs a purpose conversation by utilizing, for example, a Vision-Language Model (VLM) based on image/text understanding. A VLM may include applications through fine-tuning learning for various models including a previously announced model such as Contrastive Language-Image Pre-training (CLIP), Bootstrapping CLIP (BLIP), BLIP-2, and InstructBLIP, etc., a model to be announced in the future, etc.

A user interface device 200 may output to a user 300 first reference content provided from a server 100, and an auxiliary first system conversation that derives a user conversation related to first reference content (e.g., requests a user conversation that describes a situation within content understood by a user regarding first reference content) S320 and S325. For example, a first system conversation may be “Describe this picture.”

A first user conversation in response to a first system conversation may be transmitted to a server 100 through a user interface device 200 S330 and S325. For example, a first user conversation may be “Dad is doing the dishes. The sink is overflowing. The kids are secretly stealing cookies.”.

A second system conversation may be selected based on a first user conversation S340. A user interface device 200 may output a second system conversation provided from a server 100 to a user 300 S350 and S355. For example, a second system conversation may be “Good explanation. By the way, what is Mom doing outside the window?”.

A second user conversation in response to a second system conversation may be transmitted to a server 100 through a user interface device 200 S360 and S365. For example, a second user conversation may be “Outside the window, my mom is mowing the lawn while talking on the phone.”.

In this process, a conversation processing module 130 and a multimodal-based voice recognition module 135 of a server 100 may be involved. For example, through a multimodal-based voice recognition module 135, a user's voice may be converted into text by utilizing content presented to a user as context information. Through a conversation processing module 130, a conversation between a user and a system may be performed based on a generated conversation task.

If sufficient information has been accumulated to make a prediction about the degenerative brain function decline of a corresponding user only with exchange of first reference content, a first system conversation, and a first user conversation in response thereto, S340, S350, S355, S360 and S365 may be omitted.

Alternatively, if information accumulated through a first user conversation and a second user conversation is not sufficient to make a prediction about the degenerative brain function decline of a corresponding user, a third system conversation based on a second user conversation, and a third user conversation in response to a third system conversation (further, fourth and fifth system conversations/user conversations) may be additionally exchanged.

Based on the voice/text of user conversations accumulated in this way, a server 100 may predict a user's degenerative brain function decline S370 and generate a first prediction result. A first prediction result may be provided to a user 300 through a user interface device 200 S380 and S385.

In this process, a voice/text-based degenerative brain function decline prediction module 140 of a server 100 may be involved. For example, when a conversation between a system and a user ends, based on the voice/text of user conversations accumulated, a user's cognitive function may be scored and a determination on whether there is degenerative brain function decline may be performed, and a corresponding analysis result may be provided/fed back to a user 300.

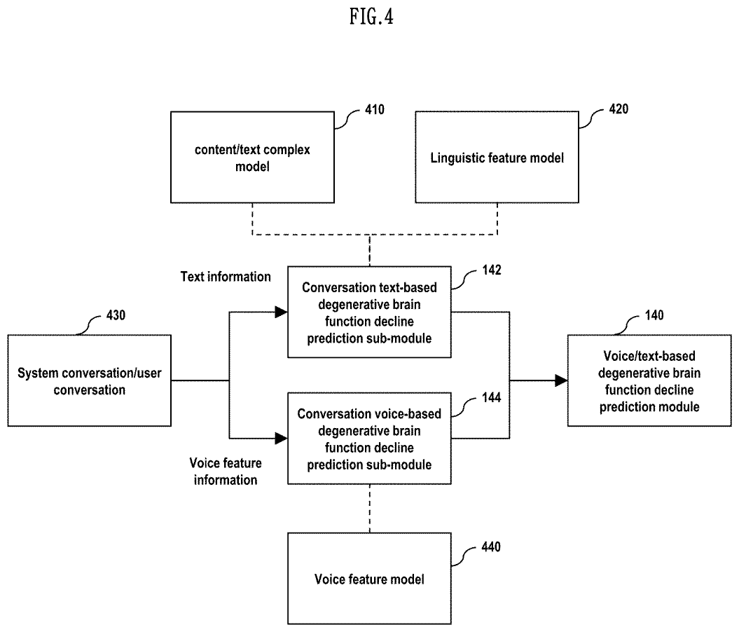

FIG. 4 shows an example of conversational voice/text-based degenerative brain function decline prediction according to the present disclosure.

A voice/text-based degenerative brain function decline prediction module 140 may perform an integrated prediction of degenerative brain function decline based on conversation text and/or conversation voice.

A conversation text-based degenerative brain function decline prediction sub-module 142 may utilize a content/text complex model 410 and/or a linguistic feature model 420.

A content/text complex model 410 is intended to distinguish between a patient group with degenerative brain function decline and a normal group based on a VLM described by referring to FIG. 3, and it may correspond to a model learned based on multimodal data consisting of content (e.g., an image) and/or text. Degenerative brain function decline may be predicted by applying a patient group model and a normal group model of a content/text complex model 410 to text information of a system conversation and/or a user conversation 430. For example, among the results of comparing text information of a system conversation and/or a user conversation 430 for specific reference content with each of a patient group model and a normal group model of a content/text complex model 410 based on the same specific reference content, according to whether it is more similar to a patient group model or a normal group model, whether a corresponding user has degenerative brain function decline may be determined.

A linguistic feature model 420 may correspond to a model learned based on linguistic feature data that distinguishes a patient group with degenerative brain function decline and a normal group appearing in text. Degenerative brain function decline may be predicted by applying a patient group model and a normal group model of a linguistic feature model 420 to text information of a system conversation and/or a user conversation 430. For example, among the results of comparing text information of a system conversation and/or a user conversation 430 for specific reference content with each of a patient group model and a normal group model of a linguistic feature model 420 based on the same specific reference content, according to whether it is more similar to a patient group model or a normal group model, whether a corresponding user has degenerative brain function decline may be determined.

A conversation voice-based degenerative brain function decline prediction sub-module 144 may utilize a voice feature model 440. A voice feature model 440 may also be referred to as an acoustic feature model.

A voice feature model 440 may correspond to a model learned based on voice feature data that distinguishes between a patient group with degenerative brain function decline and a normal group appearing in voice data. Degenerative brain function decline may be predicted by applying a patient group model and a normal group model of a voice feature model 440 to voice feature information of a system conversation and/or a user conversation 430. For example, among the results of comparing voice feature information of a system conversation and/or a user conversation 430 for specific reference content with each of a patient group model and a normal group model of a voice feature model 410 based on the same specific reference content, according to whether it is more similar to a patient group model or a normal group model, whether a corresponding user has degenerative brain function decline may be determined.

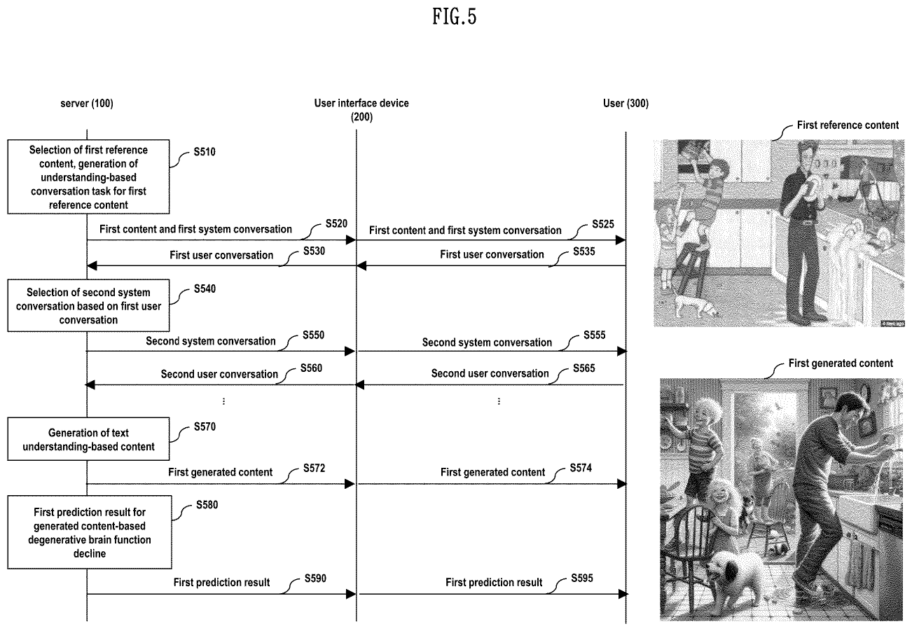

FIG. 5 shows an example of a generated content-based degenerative brain function decline prediction operation according to the present disclosure.

Since S510, S520, S525, S530, S535, S540, S550, S555, S560 and S565 in FIG. 5 may be performed in the same way as S310, S320, S325, S330, S335, S340, S350, S355, S360 and S365 in an example of FIG. 3, an overlapping description is omitted.

If information accumulated through at least one user conversation is sufficient to predict degenerative brain function decline of a corresponding user (e.g., if a conversation including a description of a user's content ends), first generated content may be generated based on text corresponding to at least one user conversation S570. First generated content may be provided to a user 300 through a user interface device 200 S572 and S574.

For example, unlike an example in FIG. 3, text corresponding to at least one user conversation in an example of FIG. 5 may be “In this picture, various things are happening in the kitchen. On the left, a young boy stands on a chair and tries to take out a bowl from the top kitchen shelf. Below it, a young blonde girl is playing with her dog. On the right, a man in jeans and a green shirt is washing dishes next to the sink. However, water overflows from the sink and fills the floor. In the background, you can see a woman hanging out the laundry in the backyard through a window.”. Accordingly, first generated content (e.g., an image) may be generated based on the text about a situation described in at least one user conversation. An image corresponding to an example of generated first generated content is shown at a bottom-right position of FIG. 3.

In this process, a text understanding-based content generation module 160 of a server 100 may be involved. For example, a text understanding-based content generation module 160 may acquire text corresponding to at least one user conversation from a multimodal-based voice recognition module 135 and generate generated content based on acquired text.

Based on first generated content, a server 100 may predict a user's degenerative brain function decline S580 and generate a first prediction result. A first prediction result may be provided to a user 300 through a user interface device 200 S590 and S595.

In this process, a generated content-based degenerative brain function decline prediction module 145 of a server 100 may be involved. For example, when a conversation between a system and a user ends, a determination on degenerative brain function decline may be performed based on first generated content generated based on text of accumulated user conversations, and first reference content presented to a user, and a corresponding analysis result may be provided/fed back to a user 300.

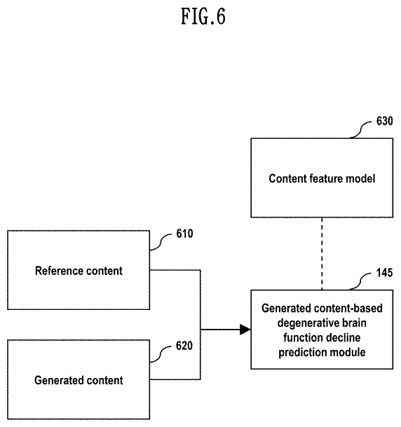

FIG. 6 shows an example of generated content-based degenerative brain function decline prediction according to the present disclosure.

A generated content-based degenerative brain function decline prediction module 145 may receive reference content 610 corresponding to content provided to a user and generated content 620 generated based on text of a user conversation. In addition, a generated content-based degenerative brain function decline prediction module 145 may utilize a content feature model 630.

A content feature model 630 may correspond to a model learned based on content data that distinguishes between a patient group with degenerative brain function decline and a normal group appearing in content. Degenerative brain function decline may be predicted by applying a patient group model and a normal group model of a content feature model 630 to reference content 610 and generated content 620 generated based on at least one user conversation for corresponding reference content. For example, among the results of comparing generated content 620 generated based on at least one user conversation for specific reference content with each of a patient group model and a normal group model of a content feature model 630 based on the same specific reference content 610, according to whether it is more similar to a patient group model or a normal group model, whether a corresponding user has degenerative brain function decline may be determined.

Additionally or alternatively, generated content-based degenerative brain function decline prediction may be performed based on comparison between a feature of generated content and a feature of reference content. For example, a feature of content may include a position, a size, a color, a sound, a distance between objects, a direction, etc. of each object in content. Accordingly, whether there is degenerative brain function decline may be determined based on similarity, etc., which is a result of comparing a feature of reference content and a feature of generated content.

In addition, a result of generated content-based degenerative brain function decline prediction may be utilized for feedback for cognitive function training/improvement, as described later.

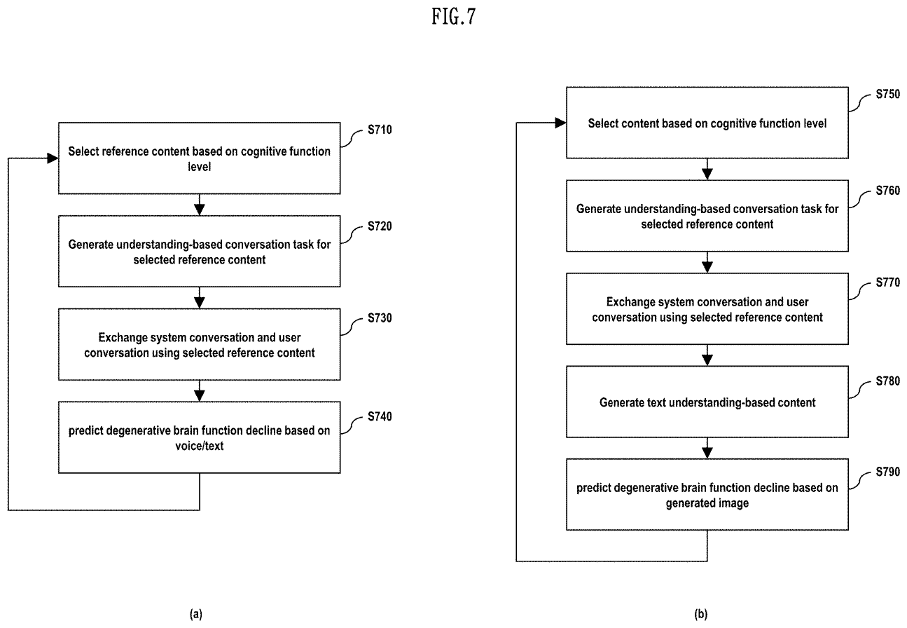

FIG. 7 shows an example of an interactive cognitive function training/improvement method using content according to the present disclosure.

FIG. 7(a) corresponds to an example of an interactive cognitive function training/improvement method based on voice/text-based degenerative brain function decline prediction according to the present disclosure.

In S710, a server 100 may select reference content based on a cognitive function level. If there is no data on a cognitive function level for a corresponding user, a default/initial value may be applied as a cognitive function level.

In this regard, an interactive cognitive function training/improvement module 150 of a server 100 may be involved. For example, an interactive cognitive function training/improvement module 150 may select content predetermined per cognitive function level of a corresponding user from content DB 110. Content corresponding to a cognitive function level may be provided to a user 300 through a user interface device 200 as reference content.

In S720, a server 100 may generate an understanding-based conversation task for selected reference content, and it corresponds to generation of a conversation task in S310 of FIG. 3, so an overlapping description is omitted. In addition, since S730 and S740 correspond to S320 to S385 in an example of FIG. 3, an overlapping description is omitted.

When a prediction result for degenerative brain function decline is generated based on voice/text in S740, a cognitive function level of a corresponding user may be determined from a generated prediction result. A determined cognitive function level may be utilized to select reference content in S710. For example, first reference content may be selected to derive a first prediction result through subsequent steps, and second reference content may be selected based on a first prediction result to derive a second prediction result through subsequent steps. In some examples, third reference content may be selected based on a second prediction result and a third prediction result may be derived through subsequent steps. Through this process, interactive cognitive function training/improvement for a corresponding user may be performed.

FIG. 7(b) corresponds to an example of an interactive cognitive function training/improvement method based on generated content-based degenerative brain function decline prediction according to the present disclosure.

In S750, a server 100 may select reference content based on a cognitive function level. If there is no data on a cognitive function level for a corresponding user, a default/initial value may be applied as a cognitive function level.

In this regard, an interactive cognitive function training/improvement module 150 of a server 100 may be involved. For example, an interactive cognitive function training/improvement module 150 may select content predetermined per cognitive function level of a corresponding user from content DB 110. Content corresponding to a cognitive function level may be provided to a user 300 through a user interface device 200 as reference content.

In S760, a server 100 may generate an understanding-based conversation task for selected reference content, and it corresponds to generation of a conversation task in S510 of FIG. 5, so an overlapping description is omitted. In addition, since S770 to S790 correspond to S520 to S595 in an example of FIG. 5, an overlapping description is omitted.

When a prediction result for degenerative brain function decline is generated based on a generated image in S790, a cognitive function level of a corresponding user may be determined from a generated prediction result. A determined cognitive function level may be utilized to select reference content in S750. For example, first reference content may be selected to derive a first prediction result through subsequent steps, and second reference content may be selected based on a first prediction result to derive a second prediction result through subsequent steps. In some examples, third reference content may be selected based on a second prediction result and a third prediction result may be derived through subsequent steps. Through this process, interactive cognitive function training/improvement for a corresponding user may be performed.

An example of FIG. 7(a) and an example of FIG. 7(b) are described as a distinct example for clarity of a description, but an example of a combination of an example of FIG. 7(a) and an example of FIG. 7(b) (e.g., sequential/parallel performance of a voice/text-based degenerative brain function decline prediction operation, and a generated image-based degenerative brain function prediction operation) is also included in a scope of the present disclosure.

Examples of the present disclosure include a variety of methods for predicting degenerative brain function decline and training/improving a cognitive function based on a conversation using various types of content provided through a user interface device (e.g., a smartphone, etc.) easily accessible to a general user. In particular, a method for exchanging a user conversation with a system conversation by understanding given reference content and automatically generating a conversation task such as Q&A, situation description, etc., and a method for generating new content based on a situation description vocalized by a user after understanding given reference content and comparing it with reference content may be applied independently or in combination. Accordingly, degenerative brain function decline prediction and cognitive function training/improvement may be performed efficiently and accurately by using content provided without substantive limits.

A component described in illustrative embodiments of the present disclosure may be implemented by a hardware element. For example, the hardware element may include at least one of a digital signal processor (DSP), a processor, a controller, an application-specific integrated circuit (ASIC), a programmable logic element such as a FPGA, a GPU, other electronic device, or a combination thereof. At least some of functions or processes described in illustrative embodiments of the present disclosure may be implemented by a software and a software may be recorded in a recording medium. A component, a function and a process described in illustrative embodiments may be implemented by a combination of a hardware and a software.

A method according to an embodiment of the present disclosure may be implemented by a program which may be performed by a computer and the computer program may be recorded in a variety of recording media such as a magnetic Storage medium, an optical readout medium, a digital storage medium, etc.

A variety of technologies described in the present disclosure may be implemented by a digital electronic circuit, a computer hardware, a firmware, a software or a combination thereof. The technologies may be implemented by a computer program product, i.e., a computer program tangibly implemented on an information medium or a computer program processed by a computer program (e.g., a machine readable storage device (e.g.: a computer readable medium) or a data processing device) or a data processing device or implemented by a signal propagated to operate a data processing device (e.g., a programmable processor, a computer or a plurality of computers).

Computer program(s) may be written in any form of a programming language including a compiled language or an interpreted language and may be distributed in any form including a stand-alone program or module, a component, a subroutine, or other unit suitable for use in a computing environment. A computer program may be performed by one computer or a plurality of computers which are spread in one site or multiple sites and are interconnected by a communication network.

An example of a processor suitable for executing a computer program includes a general-purpose and special-purpose microprocessor and one or more processors of a digital computer. Generally, a processor receives an instruction and data in a read-only memory or a random access memory or both of them. A component of a computer may include at least one processor for executing an instruction and at least one memory device for storing an instruction and data. In addition, a computer may include one or more mass storage devices for storing data, e.g., a magnetic disk, a magnet-optical disk or an optical disk, or may be connected to the mass storage device to receive and/or transmit data. An example of an information medium suitable for implementing a computer program instruction and data includes a semiconductor memory device (e.g., a magnetic medium such as a hard disk, a floppy disk and a magnetic tape), an optical medium such as a compact disk read-only memory (CD-ROM), a digital video disk (DVD), etc., a magnet-optical medium such as a floptical disk, and a ROM (Read Only Memory), a RAM (Random Access Memory), a flash memory, an EPROM (Erasable Programmable ROM), an EEPROM (Electrically Erasable Programmable ROM) and other known computer readable medium. A processor and a memory may be complemented or integrated by a special-purpose logic circuit.

A processor may execute an operating system (OS) and one or more software applications executed in an OS. A processor device may also respond to software execution to access, store, manipulate, process and generate data. For simplicity, a processor device is described in the singular, but those skilled in the art may understand that a processor device may include a plurality of processing elements and/or various types of processing elements. For example, a processor device may include a plurality of processors or a processor and a controller. In addition, it may configure a different processing structure like parallel processors. In addition, a computer readable medium means all media which may be accessed by a computer and may include both a computer storage medium and a transmission medium.

The present disclosure includes detailed description of various detailed implementation examples, but it should be understood that those details do not limit a scope of claims or an invention proposed in the present disclosure and they describe features of a specific illustrative embodiment.

Features which are individually described in illustrative embodiments of the present disclosure may be implemented by a single illustrative embodiment. Conversely, a variety of features described regarding a single illustrative embodiment in the present disclosure may be implemented by a combination or a proper sub-combination of a plurality of illustrative embodiments. Further, in the present disclosure, the features may be operated by a specific combination and may be described as the combination is initially claimed, but in some cases, one or more features may be excluded from a claimed combination or a claimed combination may be changed in a form of a sub-combination or a modified sub-combination.

Likewise, although an operation is described in specific order in a drawing, it should not be understood that it is necessary to execute operations in specific turn or order or it is necessary to perform all operations in order to achieve a desired result. In a specific case, multitasking and parallel processing may be useful. In addition, it should not be understood that a variety of device components should be separated in illustrative embodiments of all embodiments and the above-described program component and device may be packaged into a single software product or multiple software products.

Illustrative embodiments disclosed herein are just illustrative and do not limit a scope of the present disclosure. Those skilled in the art may recognize that illustrative embodiments may be variously modified without departing from a claim and a spirit and a scope of its equivalent.

Accordingly, the present disclosure includes all other replacements, modifications and changes belonging to the following claim.

What is claimed:

1. A method for supporting a prediction for a degenerative brain function decline of a user, the method comprising:
outputting, to the user, through a user interface device, a first reference content, and at least one system conversation triggering at least one user conversation related to the first reference content;
receiving, from the user, through the user interface device, at least one user conversation for the first reference content; and
based on at least one of text information corresponding to the at least one user conversation for the first reference content, or voice feature information of the at least one user conversation for the first reference content, acquiring a first prediction result for the degenerative brain function decline of the user.

2. The method of claim 1, wherein the method further includes providing the first prediction result to the user through the user interface device.

3. The method of claim 1, wherein:
a first user conversation of the at least one user conversation is associated with a first system conversation of the at least one system conversation,
a second system conversation of the at least one system conversation is generated or selected based on the first user conversation.

4. The method of claim 1, wherein the at least one system conversation is generated or selected based on an understanding-based conversation task for the first reference content.

5. The method of claim 1, wherein the first prediction result is acquired based on at least one of:
an application of a patient group model and a normal group model learned based on multimodal data including a content and a text to the first reference content and the text information corresponding to the at least one user conversation for the first reference content;
an application of a patient group model and a normal group model learned based on linguistic feature data to the text information corresponding to the at least one user conversation for the first reference content; or
an application of a patient group model and a normal group model learned based on voice feature data to the voice feature information of the at least one user conversation for the first reference content.

6. The method of claim 1, wherein the method further includes:
outputting, to the user, through the user interface device, a second reference content selected based on the first prediction result for the degenerative brain function decline of the user, and at least one system conversation triggering at least one user conversation related to the second reference content;
receiving, from the user, through the user interface device, at least one user conversation for the second reference content; and
based on at least one of text information corresponding to the at least one user conversation for the second reference content, or voice feature information of the at least one user conversation for the second reference content, acquiring a second prediction result for the degenerative brain function decline of the user.

7. The method of claim 6, wherein the method further includes providing the second prediction result to the user through the user interface device.

8. The method of claim 1, wherein the content includes at least one of an image, a video, a text, a voice, or a sound.

9. A device for supporting a prediction for a degenerative brain function decline of a user, the device comprising:
a memory;
a transceiver; and
a processor,
wherein the processor is configured to perform the method according to claim 1.

10. A method for supporting a prediction for a degenerative brain function decline of a user, the method comprising:
outputting, to the user, through a user interface device, a first reference content, and at least one system conversation triggering at least one user conversation related to the first reference content;
receiving, from the user, through the user interface device, at least one user conversation for the first reference content; and
based on a first generated content generated based on the at least one user conversation for the first reference content, acquiring a first prediction result for the degenerative brain function decline of the user.

11. The method of claim 10, wherein the method further includes providing the first prediction result to the user through the user interface device.

12. The method of claim 10, wherein:
a first user conversation of the at least one user conversation is associated with a first system conversation of the at least one system conversation,
a second system conversation of the at least one system conversation is generated or selected based on the first user conversation.

13. The method of claim 10, wherein the at least one system conversation is generated or selected based on an understanding-based conversation task for the first reference content.

14. The method of claim 10, wherein the method further includes outputting, to the user, through the user interface device, the first generated content generated based on the at least one user conversation for the first reference content.

15. The method of claim 10, wherein the first prediction result is acquired through an application of a patient group model and a normal group model learned based on content data to the first reference content, and the first generated content generated based on the at least one user conversation for the first reference content.

16. The method of claim 10, wherein the method further includes:
outputting, to the user, through the user interface device, a second reference content selected based on the first prediction result for the degenerative brain function decline of the user, and at least one system conversation triggering at least one user conversation related to the second reference content;
receiving, from the user, through the user interface device, at least one user conversation for the second reference content; and
based on a second generated content generated based on the at least one user conversation for the second reference content, acquiring a second prediction result for the degenerative brain function decline of the user.

17. The method of claim 16, wherein the method further includes providing the second prediction result to the user through the user interface device.

18. The method of claim 16, wherein the method further includes outputting, to the user, through the user interface device, the second generated content generated based on the at least one user conversation for the second reference content.

19. The method of claim 10, wherein the content includes at least one of an image, a video, a text, a voice, or a sound.

20. A device for supporting a prediction for a degenerative brain function decline of a user, the device comprising:
a memory;
a transceiver; and
a processor,
wherein the processor is configured to perform the method according to claim 10.
